(19) United States
(12) Patent Application Publication
(10) Pub. No.: US 2022/0102899 A1
(43) Pub. Date: Mar. 31, 2022

(54) CONNECTOR FOR CABLES

(71) Applicants: UNIVERSITE GRENOBLE ALPES, Saint Martin d'Heres (FR); INSTITUT POLYTECHNIQUE DE GRENOBLE, Grenoble (FR); CENTRE NATIONAL DE LA RECHERCHE SCIENTIFIQUE, Paris (FR)

(72) Inventors: Celine FOUARD, Corenc (FR); Jeremy LENFANT, La Tronche (FR); Gokularajah GANESARATNAM, Limoges (FR); Nikolai HUNGR, La Tronche (FR)

(21) Appl. No.: 17/413,431

(22) PCT Filed: Dec. 11, 2019
(86) PCT No.: PCT/EP2019/084752

§ 371 Date: Jun. 11, 2021

(30) Foreign Application Priority Data
Dec. 12, 2018 (FR) 1872802

(51) Int. Cl. H01R 13/436; H01R 13/629

(52) U.S. Cl. CPC H01R 13/4364; A61B 34/71; H01R 13/629

(57) ABSTRACT

The disclosure relates to the field of cabled robots and more particularly a connector for cables including a first block and a second block, the first and second blocks being configured to be connected together and to form, when they are connected together, a plurality of sliding guides in each of which a pair of first and second cables connected together at their respective first ends is intended for sliding, the connector further including a first locking component mounted on the first block so as to be movable between a locking position and a release position of the set of first ends of the first cables, and further including a second locking component mounted on the second block so as to be movable between a locking position and a release position of the set of first ends of the second cables.

### TECHNICAL FIELD

The invention relates to the field of cabled robots. The invention more particularly relates to a connector for cables intended for equipping a cabled robot. It can be applied advantageously in the medical field, and in particular the field of interventional radiology, the latter consisting in particular in inserting needles into the body of a patient in order to take therefrom a sample or treat a tumour for example. However, its application can also be considered in the field of industrial robotics.

### PRIOR ART

In reference to FIG. 1, a cabled robot 9 can be defined as a type of parallel manipulator wherein flexible cables 91, such as Bowden cables, are used as actuators. More particularly, an end of each cable can be wound around a rotor driven in rotation by a motor 92 and the other end can be connected to an instrument or instrument element 93, designed to interact with the environment. In the medical field, the instrument 93 can be a surgical tool, more or less complex, and the environment is the body of the patient 8. The instrument 93 is thus directed towards the cables 91 due to the modulation, in particular in length, in tension and/or in torsion, of the properties of the cables by actuating at least one motor 92.

The actuating of the motor or motors 92 can itself be controlled by a control box 94. In the medical field, the control box 94 is, for example, placed under the control of a radiologist. Generally, a connector for cables makes it possible to connect cables together, potentially in a large number, so as to use them in particular as actuators of at least one remote instrument of the connector. Generally, such a connector makes it possible to facilitate the storage of the cabled robot and makes it possible if needed to clean/sterilise only the portion of the robot that extends between the connector and the instrument, which in turn makes it possible to increase the capacity factor of the other portion of the robot.

In the medical field, it is expected that a surgical tool, as a medical instrument, has to effectively be cleaned and sterilised before each use. When the surgical tool is a complete robot, this is difficult to carry out because the robot has to be able to completely enter, including its motors, its control box, etc., the sterilisation tool. Furthermore, when certain components of the robot are not compatible with the sterilisation method, a way must be found to disconnect the portion of the robot, which can be sterilised or is to be sterilised, because having been or being intended for being put into contact with the patient, of the portion that is incompatible with the sterilisation. This must more preferably be carried out very simply, rapidly and unambiguously, in order to allow the personnel of the hospital to do so easily and to guarantee the safety of the next patient. Moreover, being able to disconnect certain portions of the robot makes it possible to achieve simpler storage, better compactness and an installation of the robot this is much simpler and much faster.

There are several connectors for cables developed for the aforementioned purposes. Such a connector for cables is for example known from document U.S. Patent 2006/0052664 A1. The connector described in this document comprises:

a housing;

a first connecting portion inside the housing; and

a second connecting portion for releasable coupling to the first connecting portion.

The first connecting portion comprises at least one guide path, a movable carriage in relation to the at least one guide path and at least one engaging apparatus, a force transmission element fastened to the carriage being coupled to a force generator. The second connecting portion comprises at least one guide path, a movable carriage in relation to the at least one guide path and at least one engaging apparatus, a force transmission element fastened to the carriage being coupled to a medical instrument. The housing provides an opening configured to receive the second connecting portion in a first direction to place the at least one engaging apparatus of the carriage of the second connecting portion in alignment with the at least one engaging apparatus of the carriage of the first connecting portion, and the connector further comprises an engaging device to displace at least one of the first and second connecting portions in a second direction, transversal to the first direction, to engage in releasable locking the engaging apparatuses aligned with carriages of the first and second portions. In this way, a subsequent linear movement of the carriage of the first connecting portion in relation to the at least one guide path of the first connecting portion results in a linear movement of the carriage of the second connecting portion in relation to the at least one guide path of the second connecting portion.

The connector according to patent document US 2006/0052664 A1 is particularly complex, although essentially intended for sequentially using simple medical instruments, such as endoscopes. On the one hand, this type of medical instruments a priori does not require any particular calibration. On the other hand, using a medical instrument of this type can follow with the use of another, clean and sterilised, even protected by a protective film, without requiring washing/sterilising the portion of the robot extending from the connector to the means for engaging the medical instrument; in other words, a simple replacing of the medical instrument used previously by a corresponding medical instrument can suffice in this context.

Moreover, when the instrument, and in particular the surgical tool, is complex to the point of requiring a particular calibration, even very precise, it would be highly appreciated to be able to retain the calibration from one use to another (for example from one patient to another), so as to effectively preserve the precision of the robot and thus ensure the safety of the patient.

Such a complex surgical tool is for example known under the name “Light Puncture Robot”. This is a robot that assists in interventional radiology. It is intended for intervening in the abdomino-pelvic zone of the patient. At least one portion of the robot is intended for being carried directly by the patient by being placed on the abdomino-pelvic zone of the patient. This robot has a parallel architecture and is set into motion using more than ten cables, preferably using fourteen cables, connected to piezoelectric motors. Thus, it is possible to offset the motors from the abdomino-pelvic zone of the patient and therefore lighten the weight of the portion of the robot directly carried by the patient.

Such a cabled robot therefore has the advantage of being light in relation to other robots. In return, it is necessary to offset the elasticity of the cables, which requires a precise calibration of the robot in order to guarantee goof precision for it. In the case of the “Light Puncture Robot”, the calibration consists in precisely measuring the positions of the robot after displacements at a given cable tension, and then comparing the errors measured using a dedicated algorithm. This calibration takes place after the manufacture of the robot and is carried out again after each maintenance operation of the robot.

Thus, the need to provide a connector that makes it possible to disconnect the instrument from its motors so as to be displaced, stored and sterilised, while still retaining the calibration of the robot, is understood better. Firstly, the safety of the patient is at stake; therefore the tension of use of the cables is to be retained concerning the precision of the robot. For the same reason, the use of the connector has to be able to be repeated and the mounting thereof without any ambiguity. Finally, to comply with usage time constraints (anaesthesia of the patient, usage time of an imaging device, etc.), it is preferable to be able to connect multiple cables simultaneously, and this simply and rapidly.

Finally, generally and for many medical applications, a robot is expected that has the particularity of being compatible with the joint use of a Magnetic Resonance Imaging (MRI) device.

An object of the present invention is therefore to propose a connector for cables that overcomes at least the aforementioned disadvantages and/or that responds to at least one of the aforementioned expectations.

The other objects, characteristics and advantages of the present invention shall appear when examining the following description and the accompany drawings. It is understood that other advantages can be incorporated.

### SUMMARY

To achieve this objective, according to an embodiment, the present invention provides a connector for cables comprising a first block and a second block, the first and second blocks being configured to be connected together and to form, when they are connected together, a plurality of sliding guides in each of which a pair of first and second cables, connected together at their respective first ends, is intended for sliding. The connector further comprises:

a first locking component mounted on the first block so as to be movable between a locking position and a release position of the set of first ends of the first cables and

a second locking component mounted on the second block so as to be movable between a locking position and a release position of the set of first ends of the second cables.

Thus, the connector makes it possible to lock the position of the first and second cables of each pair, in particular when their first respective ends are disconnected from each other and that the first and second blocks are disconnected from each other. Therefore, the connector makes it possible to retain the tension of use of each pair of cables, during successive connections and disconnections of the connector. Thus, it is not necessary for the user to again calibrate a cabled robot that would comprise for example a rotor and an instrument or instrument element connected together via the connector such as introduced hereinabove.

Optionally, the invention can further have at least any of the following characteristics:

According to a characteristic, the first locking component is configured to mechanically lock all of the first ends of the first cables simultaneously by passing from its release position to its locking position and to mechanically release all of the first ends of the first cables simultaneously by passing from its locking position to its release position, and the second locking component is configured to mechanically lock all of the first ends of the second cables simultaneously by passing from its release position to its locking position and to mechanically release all of the first ends of the second cables simultaneously by passing from its locking position to its release position.

According to another characteristic, the sliding guides are substantially straight.

According to another characteristic, the sliding guides are substantially parallel to one another.

Each block can have at least two through-bores and a connection face to the other block, each through-bore of the first block opening by a first mouth from the connection face of the first block, each through-bore of the second block opening by a first mouth from the connection face of the second block, the first mouth of each one of said at least two through-bores of the first block being configured to be placed face to face with the first mouth of a respective one of said at least two through-bores of the second block, in such a way as to form said plurality of sliding guides when the first and second blocks are connected together. According to another characteristic, each block can have a cable outlet face, preferably opposite its connection face, and each through-bore can open via a second mouth from the cable outlet face.

Each one among the first and second locking components can comprise at least one plurality of gripping devices each configured:

to engage the first end of one corresponding among the first and second cables, respectively, when the locking component is in its locking position, and

to release the first end of one corresponding among the first and second cables, respectively, when the locking component is in its release position.

The gripping devices of each locking component can be fixed with respect to their locking component and each locking component is configured to be moved in such a way as to induce the same movement of each one of its gripping devices. In this way, the first ends of ones among the first and second cables are simultaneously engaged when the locking component passes from its release position to its locking position and the first ends of ones among the first and second cables are simultaneously released when the locking component passes from its locking position to its release position. The gripping devices being fixed with respect to their locking component, their movement follows by construction the movement of their locking component. Thus, they make it possible to grip or release the first ends of the first and second cables simultaneously. Furthermore, the locking components are thus robust by design.

The connector can further comprise a plurality of pistons each intended for being fastened to the first end of a cable, each piston fastened to the first end of the first cable of a pair of cables being configured to engage a piston fastened to the first end of the second cable of the pair. Each sliding guide is configured in such a way that the first and second cables of the pair intended for sliding therein slide therein, preferably narrowly, at least by their pistons engaged together.

Each one among the first and second blocks are configured to be connected together by a relative connection movement of one with respect to the other, preferably by a relative movement of the connection face of one with respect to the connection face of the other, the pistons fastened to the first ends of the first cables and the pistons fastened to the first ends of the second cables being configured to be connected, in pairs, according to said relative movement, when the first and second locking components are in the locking position. The connector thus makes it possible, in the same movement, to connect both the first and second blocks together and each pair of cables together by their pistons when the first and second locking components are in the locking position. This makes it possible to guarantee substantial simplicity and substantial rapidity of use of the connector, and consequently of the cabled robot.

According to another characteristic, the first locking component is configured pour alternatively lock and release all the pistons fastened to the first ends of the first cables and wherein the second locking component is configured to alternatively lock and release all the pistons fastened to the first ends of the second cables. The connector thus allows for a robust locking of the first ends of the first and second cables by engaging the pistons rather than the cables themselves, the latter being moreover more likely to be used by successive connections and disconnections.

At least one piston among the two pistons of a pair can comprise on its outer periphery a lug extending substantially perpendicularly to the longitudinal direction of the sliding guide wherein it is configured to slide, and each sliding guide can comprise a groove extending continuously along the longitudinal direction of the sliding guide from the first block to the second block, when the first and second blocks are connected together, each groove being configured to guide the sliding of a lug. The connector thus makes it possible to prevent any rotation of the pistons in the sliding guides. The retaining of the orientation of the pistons is thus ensured, which is particularly advantageous, not only in terms of the service life of the cables and in terms of retaining the calibration of the robot, but also in terms of the definition of the connection mechanism of the pistons of the first and second cables together, such as shall appear in light of certain characteristics introduced hereinbelow. Each piston can have a substantially cylindrical shape. The axis of symmetry of the cylindrical shape of each piston is more preferably confounded with the axis of the sliding guide wherein the piston is intended for sliding.

In the connector according to the preceding characteristic, each through-bore can comprise, over a portion only of its extent, a portion of the groove of the sliding guide that it forms when the first and second blocks are connected together. In this way, the lug of each piston abuts, by construction, against an end of the groove of the sliding guide located as close as possible to the second end of the corresponding through-bore. Therefore, the sliding of the pistons in the sliding guides is limited in such a way that the pistons cannot be extracted from the first and second blocks from the second mouth of the through-bores. The sliding amplitude of the pistons in the sliding guides and consequently the movement amplitude of the cabled robot is limited; however, it is possible to increase practically at will the dimension of each block according to a direction substantially parallel to the longitudinal axes of the through-bores. Moreover, the pistons of a pair can be configured to cooperate together by a fastening mechanism with annular groove and collar. Among the pistons each comprising a collar, the collars are preferably configured according to the same specific orientation, for example opposite, in relation to the lugs of these pistons. Furthermore, the first and second locking components can be configured to lock the first ends of the first and second cables respectively in such a way that each piston comprising an annular groove protrudes at least by its annular groove of the first and second blocks respectively. But encore, each first mouth, configured to be placed face to face with a mouth from which the annular groove of a piston protrudes when the first and second locking components are in the locking position, can have a transverse section with bayonet fastening of the annular groove of the piston. Finally, the grooves of the sliding guides can be arranged according to the same orientation. Thus, said relative movement of the first block with respect to the second block is a translation movement of one with respect to the other, and more particularly a translation movement of the connection face of one bearing on the connection face of the other, which makes it possible to insert the annular groove of each piston of a pair into the collar of each piston of the pair.

According to another characteristic, the annular groove of each piston extends over the outer periphery of each piston according to a symmetry of rotation, even according to a symmetry of revolution.

Each one among the first and second blocks can be configured to be connected together by a relative connection movement of one with respect to the other, preferably by a relative movement of the connection face of one with respect to the other, and the first block comprises at least one positioning shape configured to cooperate with a corresponding positioning shape of the second block. The positioning shapes of the first and second blocks cooperate together by a male-female adjustment mechanism configured to guide the relative connection movement of the first and second blocks together.

Each one among the first and second blocks can be configured to be connected together by a relative connection movement of one with respect to the other, preferably by a relative movement of the connection face of one with respect to the connection face of the other, and one among the first and second blocks comprises a positioning pin configured to cooperate with a drilling comprised by the other among the first and second blocks, in such a way as to guide the relative connection movement of the first and second blocks together. The positioning pin and the drilling can cooperate together by a bayonet fastening mechanism that is equivalent, at least in terms of fastening movement amplitude and orientation, to the fastening mechanism with annular groove and collar according to which the pistons of a pair cooperate together.

Each one of the first and second blocks can comprise a protuberance, the protuberance of the first block being in contact with the protuberance of the second block when the blocks are connected together, and the first locking component is mounted on the protuberance of the first block so as to be movable in translation and the second locking component is mounted on the protuberance of the second block so as to be movable in translation. Furthermore, each protuberance can comprise two lateral sides and two longitudinal sides and each locking component can comprise at least one longitudinal portion and first and second lateral portions connected together by said at least one longitudinal portion. The protuberance of the first block is configured in such a way as to limit the translation movement of the first locking component to a continuum of positions comprised between its locking position and its release position by abutment of one among the first and second lateral portions of the first locking component on one of the two lateral sides of the protuberance of the first block and by abutment of the other among the first and second lateral portions of the first locking component on the other of the two lateral sides of the protuberance of the first block. The protuberance of the second block is configured in such a way as to limit the translation movement of the second locking component to a continuum of positions comprised between its locking position and its release position by abutment of one among the first and second lateral portions of the second locking component on one of the two lateral sides of the protuberance of the second block and by abutment of the other among the first and second lateral portions of the second locking component on the other of the two lateral sides of the protuberance of the second block. According to a characteristic each longitudinal portion of each locking component is configured to slide on one of the two longitudinal sides of the protuberance limiting its movement. Furthermore, each longitudinal portion of each locking component and each longitudinal side of the protuberance whereon the longitudinal portion is configured to slide are configured to cooperate together in such a way as to allow for the relative sliding thereof, while still engaging them. This cooperation is for example carried out by at least one mechanism with a slide block and slide and/or a mechanism with a lug and groove.

The connector can further comprise at least two return members, a first return member configured to exert on the first locking component a return force tending to maintain the first locking component in one of its locking and release positions, a second return member configured to exert on the second locking component a return force tending to maintain the second locking component in one of its locking and release positions. Furthermore, the connector can comprise two latches:

a first latch mounted on the first block so as to be movable between a retracted position and a deployed position, the first latch being configured jointly with the first locking component to oppose the return force that the first return member exerts on the first locking component in such a way as to maintain the first locking component in the other of its locking and release positions when the first latch is in its deployed position, and to not oppose the return force that the first return member exerts on the first locking component when the first latch is in its retracted position, and

a second latch mounted on the second block so as to be movable between a retracted position and a deployed position, the second latch being configured jointly with the second locking component to oppose the return force that the second return member exerts on the second locking component in such a way as to maintain the second locking component in the other of its locking and release positions when the second latch is in its deployed position, and to not oppose the return force that the second return member exerts on the second locking component when the second latch is in its retracted position.

In a connector according to the two preceding paragraphs, the first latch can be configured to be, in its deployed position, partially pinched between one of the lateral sides of the protuberance of the first block and the first lateral portion of the first locking component abutting on this lateral side when the first latch is in the retracted position and the first return member can be arranged between the other of the lateral sides of the protuberance of the first block and the second lateral portion of the first locking component opposite the first lateral portion of the first locking component. Moreover, the second latch can be configured to be, in its deployed position, partially pinched between one of the lateral sides of the protuberance of the second block and the first lateral portion of the second locking component abutting on this lateral side when the second latch is in the retracted position and the second return member can be arranged between the other of the lateral sides of the protuberance of the second block and the second lateral portion of the second locking component opposite the first lateral portion of the first locking component.

In a connector according to any of the three preceding paragraphs, each groove opens in such a way that each lug extends by its distal end beyond the groove configured to guide the sliding thereof, and said at least one longitudinal portion of each locking component comprises one among said at least one plurality of gripping devices, each gripping device being configured to lock the distal end of a corresponding lug so as to prevent the sliding thereof in its groove when the locking component is in the locking position. Each gripping device can comprise a removal of material made in said at least one longitudinal portion of the locking component and having a first portion forming a secondary sliding groove of the distal end of the corresponding lug when the locking component is in the release position and a second portion forming a housing open only onto the secondary groove to house the distal end of the corresponding lug when the locking component is in the locking position. Each secondary groove can be configured to be aligned with a sliding guide when the locking component is in the release position. Moreover, each housing can have, in a longitudinal section plane of the longitudinal portion of the locking component, a flared shape section opening onto the secondary groove via its widest portion, the latter having a width greater than a diameter of the distal end of the lug that it houses when the locking component is in its locking position. The locking of the distal portions of the lugs by the locking component thus configured benefits from a certain degree of freedom relative to the position of the distal end of the lug in the secondary groove. Consequently, the locking position of the first ends of the cables by the locking components benefits from the same degree of freedom.

Each block can comprise a fastening device to the other block, the fastening device of the first block being configured to removably engage the fastening device of the second block, in such a way as to maintain the first and second blocks connected together, at least when the first ends of the cables are connected in pairs and the locking components are in their release position.

According to another characteristic, each block can comprise a second block portion and a third block portion removably fastened together, each second block portion comprising a downstream portion of a first plurality of sliding guides and each third block portion comprising an upstream portion of a second plurality of sliding guides. One of the second and third block portions can comprise the protuberance of the block and each locking component can comprise two longitudinal portions, each one connected to opposite ends of the lateral portions of the locking component. A first longitudinal portion comprises the gripping devices of the first ends of the cables intended for sliding in the first plurality of sliding guides and a second longitudinal portion comprises the gripping devices of the first ends of the cables intended for sliding in the second plurality of sliding guides. The connector thus makes it possible to connect together twice as many pairs of cables together in a compact, simple and robust manner.

Another aspect of the present invention relates to a cabled robot comprising a connector for cables such as introduced hereinabove.

### BRIEF DESCRIPTION OF THE FIGURES

The purposes, objects, as well as the characteristics and advantages of the invention shall appear better in the detailed description of an embodiment of the latter which is shown in the following accompanying drawings wherein:

FIG. 1 shows a putting into situation, in the medical field, of a cabled robot that can be equipped with a connector for cables according to the invention;

FIG. 2A diagrammatically shows a connector for cables according to an embodiment of the present invention wherein the two blocks are disconnected from each other and the cables are locked relatively to the blocks;

FIG. 2B diagrammatically shows the connector for cables shown in FIG. 2A of which the blocks were connected together to define a plurality of sliding guides wherein the cables connected together in pairs are free to slide;

FIG. 3 shows a perspective view of a connector for cables according to an embodiment of the invention and of cables that this connector makes it possible to connect together in pairs;

FIG. 4 shows a perspective view of the first block, of the first locking component and of the pistons with annular groove of the connector shown in FIG. 3;

FIG. 4A shows a perspective and exploded view of the first block, of the first locking component and of the pistons with annular groove shown in FIG. 4;

FIG. 4B shows a perspective view of one of the components shown in FIG. 4A according to another orientation;

FIG. 5 shows a perspective view of the second block, of the second locking component and of pistons with collar of the connector shown in FIG. 3;

FIG. 6 is a perspective and cut-away view of any of the two locking components of the connector shown in FIG. 3;

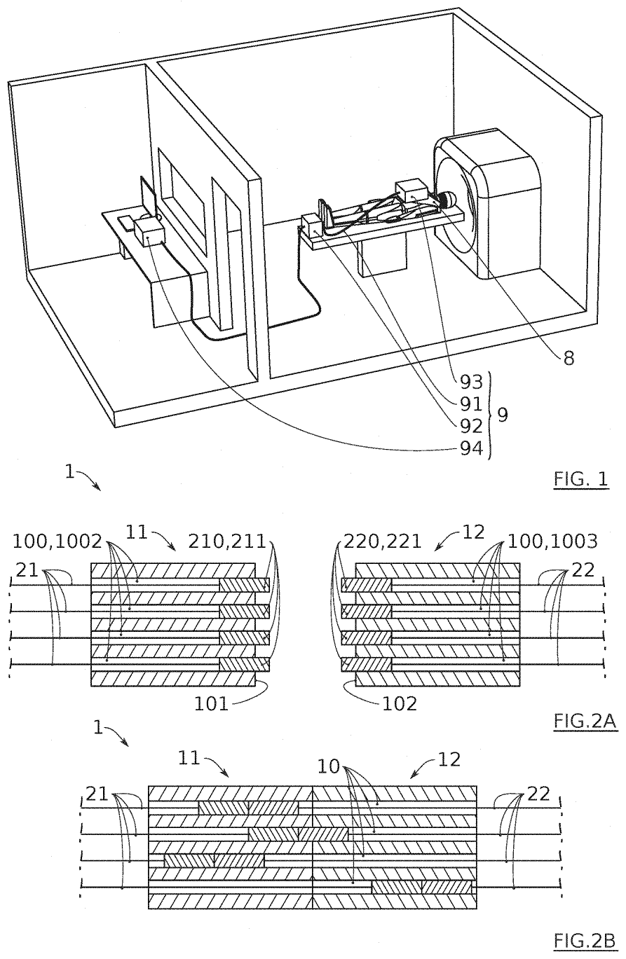

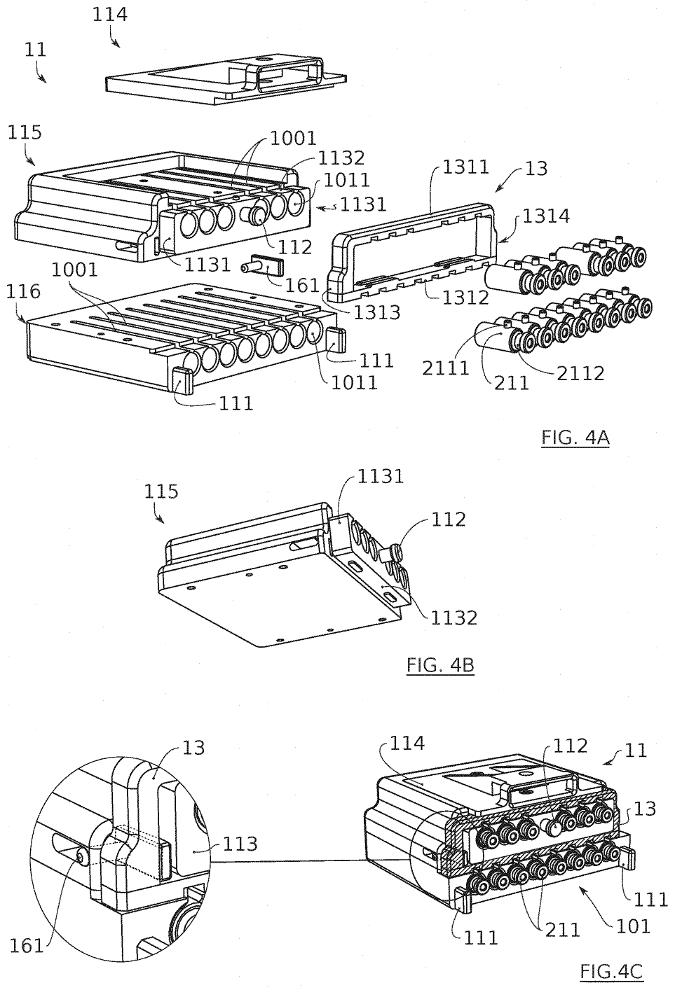

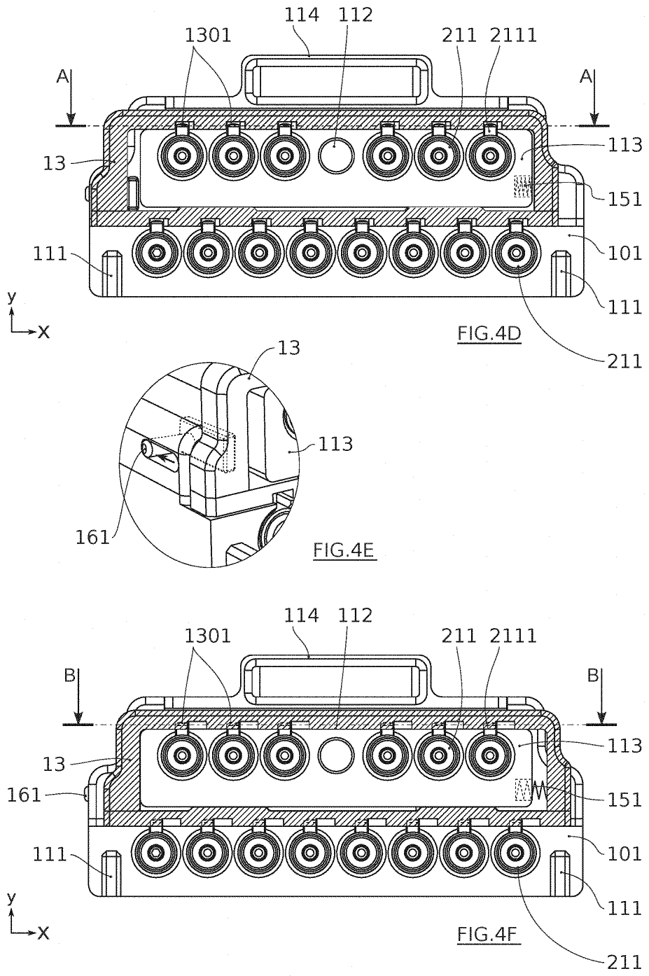

FIG. 4C takes FIG. 4 and adds thereto an enlargement focussed on a portion of this FIG. 4 showing as a cut-away a latch in the deployed position;

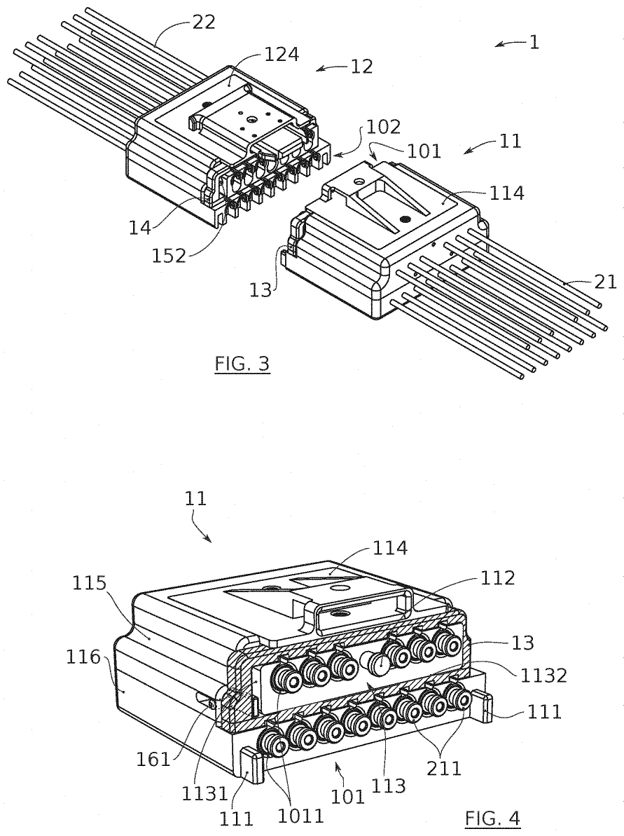

FIG. 4D is a front view of the first block shown in FIG. 3 when the cables are released;

FIG. 7 is a cross-section view, taken according to the cutting plane A-A such as shown in FIG. 4D, showing the locking/release mechanism of the cables according to an embodiment of the invention;

FIG. 4E shows as a cut-away the latch of the connector shown in FIG. 4, the latch being in the retracted position;

FIG. 4F is a front view of the first block shown in FIG. 3 when the cables are locked;

FIG. 8 is a cross-section view, taken according to the cutting plane B-B such as shown in FIG. 4F, showing the locking/release mechanism of the cables according to an embodiment of the invention;

FIG. 9 is a perspective view of the two blocks of the connector shown in FIG. 3, the blocks being connected together;

FIG. 10 is a perspective view of the ends of a pair of cables equipped with pistons of the connector according to an embodiment of the of the invention; and

FIGS. 11A to 11C are cross-section views showing the fastening mechanism with annular groove and collar according to which the pistons of the connector, according to an embodiment of the invention, cooperate together.

The drawings are given as examples and do not limit the invention. They form block diagrams intended for facilitating the understanding of the invention and are not necessarily to the scale of the practical applications.

### DETAILED DESCRIPTION

The present invention relates, according to its first aspect, to a connector for cables, and in particular for flexible cables, intended for making it possible to very quickly and easily connect a large quality of cables together. The connector according to the invention aims more particularly to connect together, on the one hand an instrument or an instrument element, on the other hand a motorised rotor assembly, of a cabled robot. Thus, according to the second aspect, the invention relates to a cabled robot comprising a connector according to the first aspect of the invention. Storage/displacement and/or cleaning/sterilisation of the cabled robot is greatly facilitated due to the fact that it comprises the connector according to the first aspect of the invention.

Regarding the connector according to the first aspect of the invention, it comprises a first block, constituting for example a male portion of the connector, and a second block, constituting for example a female portion of the connector. The male portion and the female portion of the connector according to the first aspect of the invention are configured to interlock in such a way as to simultaneously connect all the cables in pairs.

The connector, according to the first aspect of the invention, essentially differentiates itself from the connectors according to the prior art in that it makes it possible to retain a tension of use of the cables, during successive connections and disconnections of the first and second blocks from each other. Therefore, the user of the cabled robot does not have to calibrate the robot following a disconnection of the cables from each other via the connector for cables according to the first aspect of the invention. As shall appear clearly in what follows, these advantages are obtained while still retaining substantial simplicity and rapidity of use of the connector for cables and consequently of the cabled robot.

The connector for cables according to the first aspect of the invention is therefore a multi-cable connector with locking and retaining of the tension of the cables.

The cables intended for being connected together via the connector for cables according to the first aspect of the invention are for example Bowden cables. Such a cable is constituted of a flexible sheath wherein a core of the cable slides. According to a preferred implementation of the invention, the connector for cables is more particularly intended for connecting cables together constituted of polyethylene braids, for example of the Spectra® or Dyneema® brand. Moreover, the connector according to the first aspect of the invention, and more particularly each one of its constituent elements, can be entirely manufactured from a material such as plastic, compatible with use in a specific medical environment, such as the environment of an MRI device of which the use requires ensuring the absence of any metal body. Thus, the connector according to the first aspect of the invention is more preferably free from elements made of metal.

The connector for cables according to a preferred embodiment of the first aspect of the invention shall now be described in reference to FIGS. 2 to 11C.

As shown in FIG. 3, the connector 1 for cables 21, 22 essentially comprises a first block 11 and a second block 12 configured to be connected together, in such a way as to form a plurality of sliding guides 10 (Cf. FIG. 2) in each of which a pair of first and second cables 21, 22, connected together via one of their respective ends, is intended for sliding. The connector 1 also comprises a first locking component 13 and a second locking component 14. Each locking component is mounted on one of the two blocks 11 and 12 so as to be movable. Each locking component is more particularly mounted so as to be movable between two positions, a locking position and a release position of all the first ends of the first cables 21 or of the second cables 22. Each locking component is, preferably, configured to alternatively mechanically and simultaneously lock and release all of the first ends of the first cables 21 and of the seconds cables 22.

As shown in FIG. 2, and as can be seen in FIG. 3, the sliding guides are straight and parallel to one another. However, the invention is not limited to this configuration and this particular arrangement of the sliding guides, which could be elbowed and/or oriented in relation to one another according to converging or diverging directions.

Each sliding guide 10 comprises two parties, a portion referred to as upstream portion 1002, for example oriented towards the motorised assembly of the cabled robot by following the first cables 21 and a portion referred to as downstream portion 1003, for example oriented towards the instrument or instrument element to be controlled by following the second cables 22.

More particularly, still in reference to FIG. 2, each block 11, 12 has a plurality of through-bores 100 and a connection face 101, 102 to the other block. Each plurality of through-bores 100 such as shown in FIG. 2 comprises a row of four through-bores 100.

In comparison, each plurality of through-bores 100 such as shown in FIG. 3 comprises two rows of through-bores 100 superimposed between them, a first row comprising six through-bores and a second row comprising eight through-bores. Thus, the connector for cables according to the preferred embodiment of the invention makes it possible to connect together up to fourteen first and second cables 21, 22.

Described hereinbelow in more detail the cooperation between the two blocks in such a way that they are, as introduced hereinabove, configured to be alternatively connected and disconnected from each other. To do this, it is first stated that the first and second blocks 11, 12 are configured to be connected together by a relative connection movement of one with respect to the other, preferably by a relative movement of the connection face of one with respect to the connection face of the other.

In FIG. 3, a fastening device 114, 124 of the blocks 11, 12 together is shown. More particularly, the fastening device 114 of the first block 11 is configured to removably engage the fastening device 124 of the second block 12. The maintaining of the first and second blocks connected together is thus ensured, in particular when the first ends of the cables 21, 22 are connected in pairs and the locking components 13, 14 are in their release position. Here, the fastening device 114, 124 shown is a clip closure. This illustration does not however limit the invention. Alternatively, even in combination, the fastening device 114, 124 could be based on a magnetic closure mechanism with magnets, even with electromagnets. Note that the fastening device 114, 124 is preferably configured to have a certain degree of clearance between an intermediate fastening position and a final fastening position of the blocks 11, 12 together, in such a way as to accommodate a relative connection movement of the two blocks together, this relative movement being described hereinbelow. In the example of the fastening device 114, 124, by clip closure mechanism, the arch of the element 114 can have a height greater than the thickness of the clip of the element 124 so as to realise the aforementioned accommodation.

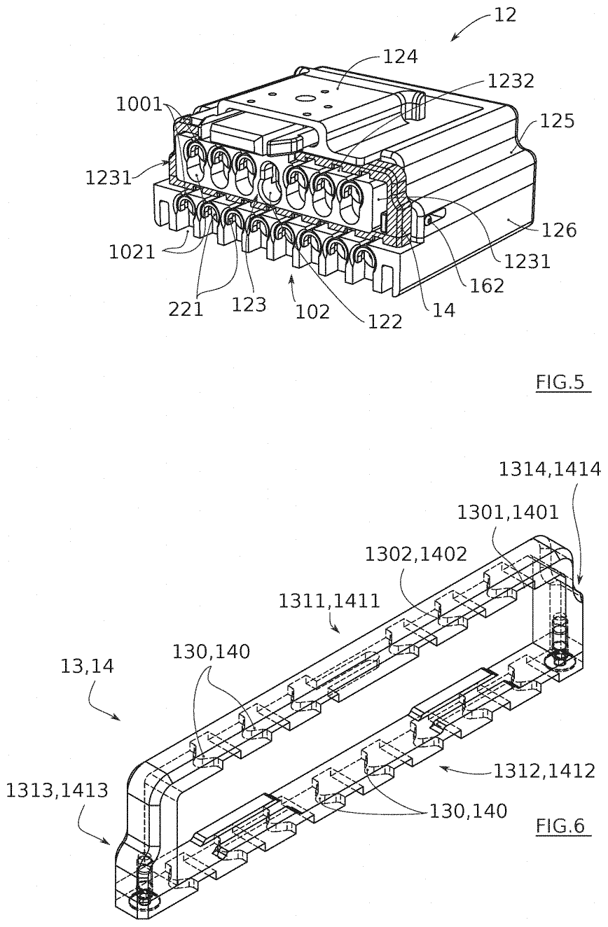

As is shown particularly well in FIGS. 4 and 5, each through-bore of the first block 11 opens by a first mouth 1011 from the connection face 101 and each through-bore of the second block 12 opens by a first mouth 1021 from the connection face 102. The first mouths 1011, 1021 are distributed on their respective connection faces 101, 102 in such a way as to form the sliding guides 10 when the first and second blocks 11, 12 are connected together.

Note, as a concern for completeness, that each connection face 101, 102 of the blocks 11, 12 can comprise an opposite from which each through-bore opens by a second mouth, this opposite face thus being a cable outlet face.

Moreover, the first block 11 can comprise at least one positioning shape 111 configured to cooperate with a corresponding positioning shape 121 of the second block 12. As shown clearly in FIGS. 3 to 5, the positioning shapes 111, 121 cooperate together by a male- female adjustment mechanism configured to guide the relative connection movement of the first and second blocks 11, 12 together. Alternatively or as a complement, the first block 11 comprises a positioning pin 112 configured to cooperate with a drilling 122 comprised by the second block 12, in such a way as to guide the relative connection movement of the first and second blocks 11, 12 together. Note here that the positioning pin 112 and the drilling 122 such as shown cooperate together by a bayonet fastening mechanism.

The relative connection movement of the two blocks 11, 12 together here is a movement that entails the sliding of the connection face 102 of the second block 12 bearing on the connection face 101 of the first block 11 in the direction and the sign of the Y axis of the coordinate system such as shown in FIGS. 4D and 4F. Obviously, the fastening device 114, 124, the positioning shapes 111, 121, and the bayonet fastening mechanism formed by the positioning pin 112 and the drilling 122 can be configured in such a way as to induce a relative connection movement of the two blocks 11, 12 together different from the one shown in the figures. However, as shall appear hereinbelow, the relative connection movement of the two blocks 11, 12 together can also be defined in such a way that the first ends of the first cables 21 are connected in pairs to the first ends of the second cables 22, simultaneously, and where applicable mechanically.

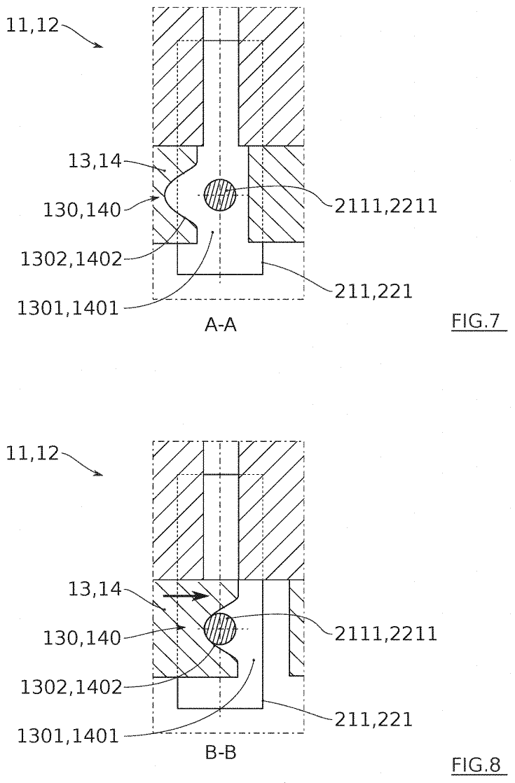

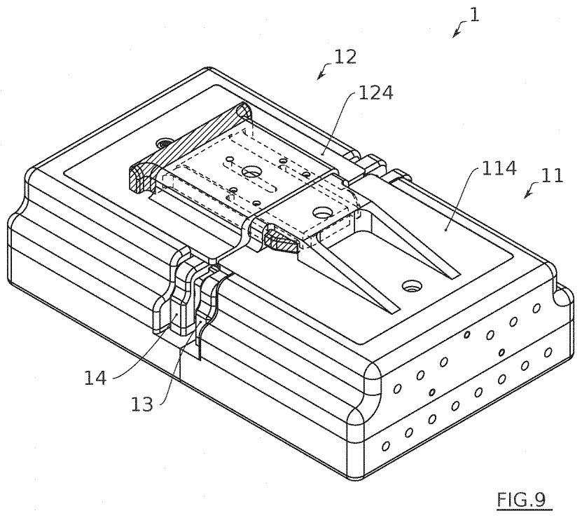

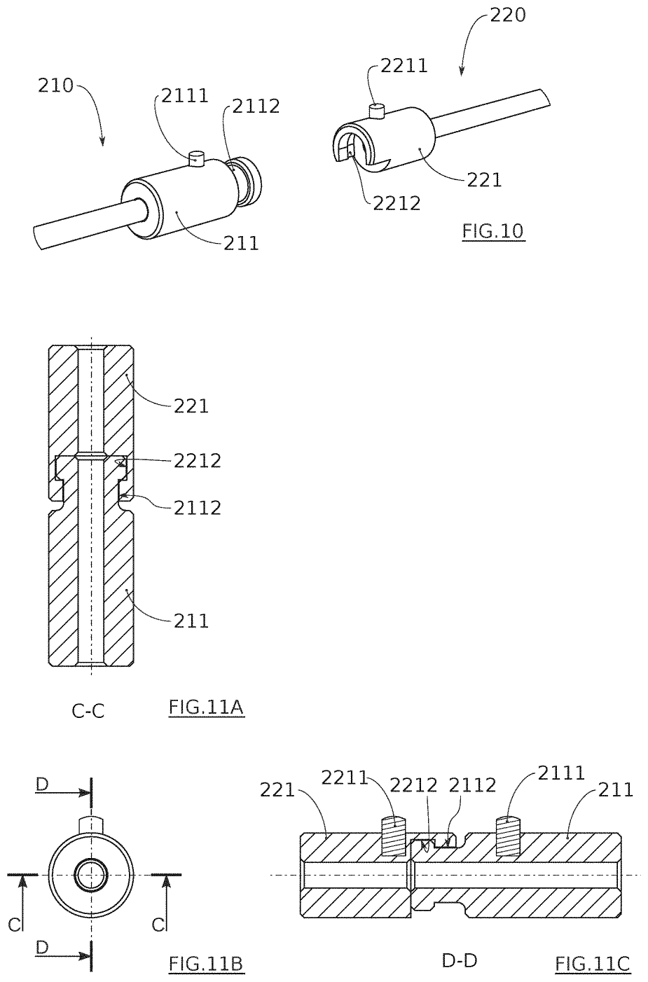

Still in reference to FIGS. 3 to 5 and in reference to FIG. 6, it appears that according to the preferred embodiment of the invention each locking component 13, 14 takes a general substantially rectangular and hollow shape configured to be moved in translation around a protuberance 113, 123 of each one of the blocks 11, 12. Moreover, according to the preferred embodiment of the invention, the locking components 13, 14 are strictly identical between them.

Described hereinbelow in more detail is the cooperation between each protuberance 113, 123 and the locking component 13, 14 mounted on this protuberance so as to be movable. Each protuberance 113, 123 comprises two lateral sides 1131, 1231 and two longitudinal sides 1132, 1232 and each locking component 13, 14 comprises two longitudinal portions 1311, 1312, 1411, 1412 and first and second lateral portions 1313, 1314, 1413, 1414 connected together by the longitudinal portions 1311, 1312, 1411, 1412. The protuberance 113 of the first block 11 is configured in such a way as to alternatively limit the translation movement of the first locking component 13:

by abutment of its first lateral portion 1313 on the lateral side 1131 of the protuberance 113 and

by abutment of its second lateral portion 1314 on the other lateral side 1131 of the protuberance 113 of the first block 11.

Furthermore, as appears clearly in light of FIGS. 4A, 4B and 6, the longitudinal portion 1311, 1312 of the first locking component 13 and each longitudinal side of the protuberance 113 of the first block 11 are configured to cooperate together in such a way as to allow for the relative sliding thereof, while still engaging them, and more particularly while still preventing the first locking component 13 from leaving the protuberance 113. This cooperation is, in the example shown, carried out on the one hand by a mechanism with a slide block and slide arranged between the longitudinal portion 1312 of the first locking component and the longitudinal side 1132 of the protuberance 113 such as referenced in FIG. 4B, on the other hand by a mechanism with a lug and groove, the lug of this mechanism extending from the longitudinal side 1132 referenced in FIG. 4A, while its groove extends over a portion of the longitudinal portion 1311 of the first locking component 13 as can be seen as a cut-away in FIG. 6.

The same applies to then cooperation between the protuberance 123 of the second block 12 and the second locking component 14.

Still concerning the cooperation between each protuberance 113, 123 and the locking component 13, 14 mounted on this protuberance so as to be movable, note that the connector further comprises two return members 151, 152 and two latches 161, 162.

In reference to FIGS. 4D and 4F, a first return member 151 is configured to exert on the first locking component 13 a return force tending to maintain the first locking component 13 in its locking position. A first latch 161 is mounted on the first block 11 so as to be movable between a retracted position (Cf. FIG. 4E) and a deployed position (Cf. the enlargement in FIG. 4C), the first latch 161 being furthermore configured together with the first locking component 13 to oppose the return force that the first return member 151 exerts on the first locking component, in such a way as to maintain the first locking component 13 in its release position when the first latch 161 is in its deployed position, and to not oppose the return force that the first return member 151 exerts on the first locking component 13 when the first latch 161 is in its retracted position.

More particularly, the first latch 161 is configured to be, in its deployed position, partially pinched between one of the lateral sides 1131 of the protuberance of the first block 11 and the first lateral portion 1313 of the first locking component 13 abutting on this lateral side when the first latch 161 is in the retracted position. But encore, the first return member 151 is arranged between the other of the lateral sides 1131 of the protuberance of the first block 11 and the second lateral portion 1314 of the first locking component 13 opposite the first lateral portion 1313 of the first locking component 13.

The same applies to the cooperation between the protuberance 123 of the second block 12, the second return member 152 (Cf. FIG. 3) and the second latch 162.

In light of the above, it appears that the cooperation between each block, and more particularly the protuberance of each block, and the locking component mounted on this block as to be movable is thus robust and compact, in such a way that a large number of manipulations of the locking components of the connector according to the first aspect of the invention are allowed without inducing any deterioration of the connector and of its function.

Note furthermore that, as shown in FIG. 4A, the first block 11 can in fact comprise several portions that are possibly removably assembled together. One of its portions is constituted of the fastening device 114, this fastening device being different from the fastening device 124 constituting a portion of the second block 12.

Moreover, the first block can further comprise a second block portion 115 and a third block portion 116. As shown, the second block portion 115 comprises the downstream portion 1002 (already introduced hereinabove) of a first plurality of sliding guides 10 and the third block portion 116 comprises the upstream portion 1003 (also introduced hereinabove) of a second plurality of sliding guides 10. Furthermore, the second block portion 115 comprises the protuberance 113 of the first block 11.

The same applies to the second block relative to its second block portion 125 and its third block portion 126. It therefore appears that the second portions of the blocks are identical between them and that the third portions of the blocks are identical between them. Therefore, the blocks differ amongst themselves only by their fastening device 114, 124. It is understood that this characteristic of the connector according to the preferred embodiment of the invention is advantageous in terms of facility and cost of manufacturing, this advantage being further accentuated by the fact that the first and second locking components 14, 13 are identical between them.

In a manner that is not necessarily limited to the preferred embodiment of the invention, each one among the first and second locking components 13, 14 comprises at least one plurality of gripping devices 130, 140 each configured:

to engage the first end 210, 220 of one corresponding among the first and second cables 21, 22, respectively, when the locking component is in its locking position, and

to release the first end 210, 220 of one corresponding among the first and second cables 21, 22, respectively, when the locking component is in its release position.

In a broader context than the preferred embodiment of the invention, the gripping devices 130, 140 can for example take the form of hooks, each hook being configured to engage a first end of one of the first and second cables 21, 22; alternatively, the gripping devices 130, 140 can for example take the form of flared notches, each notch being flared in such a way as to allow a first end of one of the first and second cables 21, 22 via its widest portion, until pinching this first end by its narrowest portion.

It will however appear clearly hereinbelow that the preferred embodiment of the invention relating to the gripping devices 130, 140 has manifest advantages on the gripping devices 130, 140 such as considered hereinabove.

Regardless of the gripping devices 130, 140 implemented, it is preferable that the gripping devices 130, 140 of each locking component 13, 14 be fastened on their locking component; in other words, it is preferable that they not be mounted on the latter so as to be movable. The robustness of the connector and therefore its service life are effectively increased. Moreover it is preferable that each locking component 13, 14 be configured to be moved in such a way as to induce the same movement of each one of its gripping devices. Thus, the first ends 210, 220 of the first and second cables 21, 22 are by construction simultaneously engaged when the locking component passes from its release position to its locking position; likewise, the first ends 210, 220 of the first and second cables 21, 22 are by construction simultaneously released when the locking component passes from its locking position to its release position. Note that this preference is compatible with each one of the embodiments of the gripping devices 130, 140 considered hereinabove, and with the gripping devices according to the preferred embodiment of the invention (Cf. infra).

Another characteristic of the connector according to a preferred embodiment is described hereinbelow which is compatible and advantageous, potentially synergistically, with the gripping devices 130, 140 considered hereinabove and with the gripping devices 130, 140 according to the preferred embodiment of the invention.

According to this other characteristic, the connector further comprises a plurality of pistons 211, 221 such as shown diagrammatically in the FIGS. 2A and 2B and in a manner correlated with the preferred embodiment of the invention in particular in the FIGS. 3 to 5. In reference to FIGS. 2A and 2B, to FIG. 3 and to FIG. 10 in particular, each piston is intended for being fastened to the first end 210, 220 of a cable 21, 22. Furthermore, each piston 211 fastened to the first end 210 of the first cable 21 of a pair of cables 21, 22 is configured to engage a piston 221 fastened to the first end 220 of the second cable 22 of the pair. By construction, it is therefore necessary that each sliding guide 10 be configured in such a way that the first and second cables 21, 22 of the pair intended for sliding therein slide therein, preferably narrowly, at least by their pistons 211, 221 engaged together.

As shown in FIG. 10, a piston 211 fastened to the first end of a first cable 21 and a piston 221 fastened to the first end of a second cable 22 can be configured to be connected according to a relative movement of one with respect to the other. According to the preferred embodiment of the invention, the pistons 211, 221 of a pair are configured to cooperate together by a fastening mechanism with annular groove and collar such as shown in FIG. 10 and FIGS. 11A, 11B and 11C. As announced hereinabove, it is then advantageous that the relative fastening movement of the pistons of a pair be equivalent to the relative connection movement of the blocks together, in such a way that, when the first and second locking components 13, 14 are in the locking position, the connection of the blocks together induces the fastening in pairs of the pistons 211 with annular groove with the pistons 221 with a collar. Thus, the connector makes it possible, in the same movement, to connect both the first and second blocks together and each pair of cables together by their pistons when the first and second locking components are in the locking position. This makes it possible to guarantee substantial simplicity and substantial rapidity of use of the connector, and consequently of the cabled robot.

However, the invention is not limited to a fastening mechanism of the pistons of each pair by a relative movement of these pistons together. Indeed, it can also be considered that the pistons cannot be connected together in pairs via a fastening mechanism with permanent magnets, even by electromagnets. According to this latter alternative, the electromagnets could be powered via the cables 21, 22 via conductive wires arranged along these cables 21, 22. Note that these alternatives also make it possible to guarantee substantial simplicity and substantial rapidity of use of the connector. Note however that, contrary to these alternatives, the pistons according to the preferred embodiment of the invention can be carried out from a non-metal material, and in particular from a plastic material, compatible in particular with the use of the cabled robot in an MRI device.

Concerning the different gripping devices considered, this latter characteristic according to which the connector 1 comprises the aforementioned pistons 211, 221, has the synergistic advantage of allowing the first locking component 13 to be configured to alternatively lock and release all the pistons 211 fastened to the first ends of the first cables 21 and that the second locking component 14 be configured to alternatively lock and release all the pistons 221 fastened to the first ends of the second cables 22. Any risk of wear of the cables is thus prevented by using, for their locking in particular, the pistons 211, 221 which are by construction more robust than the cables 21, 22.

As shown in FIG. 10 and FIG. 11C, each piston 211, 221 supports, according to the preferred embodiment of the invention, a lug 2111, 2211. Details are provided hereinbelow on how this lug is advantageously implemented to prevent any rotation of the pistons in the sliding guides 10. Note however that it is possible to prevent any rotation of the pistons in the sliding guides 10 by providing these pistons with a general non-cylindrical shape, for example a general shape with an oblong, oval or polygonal transversal section; the pistons cooperating closely with the sliding guides 10 are thus prevented from rotating on themselves in their guide. It will also be shown hereinbelow that the lug 2111, 2211 of each piston 211, 221 can further be advantageously implemented to allow for the locking of the first ends of the cables 21, 22 by the locking components 13, 14.

Each piston 211, 221 of the preferred embodiment of the invention has a general substantially cylindrical shape. The axis of symmetry of the cylindrical shape of each piston is preferably confounded with the axis of the sliding guide 10 wherein the piston is intended for narrowly sliding.

The lug 2111, 2211 of each piston 211, 221 extends more particularly from the outer periphery of the piston and substantially perpendicularly to the axis of symmetry of the cylindrical shape of the piston. Moreover, each sliding guide 10 comprises a groove 1001 extending continuously along the longitudinal direction of the sliding guide from the first block 11 to the second block 12, when the first and second blocks are connected together. Each groove 1001 is more particularly configured to guide the sliding of a lug 2111, 2211.

More particularly, each through-bore 100 comprises, on a portion only of its extent, a portion of the groove 1001 of the sliding guide 10 that it forms when the first and second blocks 11, 12 are connected together. In this way, the lug 2111, 2211 of each piston abuts, by construction, against an end of the groove 1001 located as close as possible to the second mouth of the corresponding through-bore 100. Therefore, the sliding of the pistons 211, 221 in the sliding guides 10 is limited in such a way that the pistons cannot be extracted from the first and second blocks 11, 12 from their cable outlet faces opposite their connection faces 101, 102. The sliding amplitude of the pistons 211, 221 in the sliding guides 10 and consequently the movement amplitude of the cabled robot are limited; however, it is possible to increase practically at will the dimension of each block 11, 12 according to a direction substantially parallel to the longitudinal axes of the through-bores 100, and to thus ensure a sufficient movement amplitude of the cabled robot.

As evoked hereinabove, the connector 1 thus makes it possible to prevent any rotation of the pistons 211, 221 in the sliding guides 10. The retaining of the orientation of the pistons is thus assured, which is particularly advantageous, not only in terms of the service life of the sables and in terms of retaining the calibration of the robot, but also in terms of the definition of the connection mechanism of the pistons of first and second cables together, as shall appear in light of the characteristics introduced hereinbelow.

As announced hereinabove, the pistons 211, 221 of a pair are configured to cooperate together by a fastening mechanism with annular groove and collar. Therefore, this fastening mechanism can be configured on each piston jointly with the lug of this piston 2111, 2211.

Thus, as shown in FIG. 5 in particular, among the pistons 221 comprising a collar 2212, each collar 2212 is configured in such a way that their opening is located opposite lugs 2211 of these pistons 221, and therefore opposite the grooves 1001 made in the blocks 11, 12. The annular groove 2112 of each piston 211 extends over the outer periphery of each piston 211 according to a symmetry of revolution about the axis of symmetry of the general cylindrical shape of the piston 211; it is understood that, therefore, the lug 2111 of each piston 211 can be configured according to any orientation on the outer periphery of the piston 211.

Furthermore, the first locking component 13 is configured, as shown in FIG. 4, to lock the first ends of the first cables 21 in such a way that each piston 211 comprising an annular groove 2112 protrudes at least by its annular groove from the first block 11.

Moreover, the grooves 1001 of the sliding guides 10 are arranged according to the same orientation and each first mouth 1021, configured to be placed face to face with a mouth 1011 from which the annular groove 2112 of a piston 211 protrudes when the first locking component 13 is in the locking position, has a bayonet fastening transverse section such that it allows in a first step to engage the portion of the piston 211 protruding, by a translation movement of the blocks 11, 12 together according to the axis longitudinal of the through-bores 100, and in a second step to fasten the annular groove 2112 of this piston 211 in the collar 2212 of the piston 221, by a translation movement of the blocks 11, 12 together according to the direction of the axis Y such as shown in FIGS. 4D and 4F. In other words, the relative connection movement of the blocks 11, 12 together is a translation movement of one with respect to the other, and more particularly a translation movement of the connection face 101 of one 11 bearing on the connection face 102 of the other 12, which makes it possible to insert the annular groove 2112 of each piston 211 of a pair into the collar 2212 of each piston 221 of the pair.

As announced hereinabove, the lug 2111, 2211 of each piston 211, 221 can further be advantageously implemented to allow for the locking of the first ends of the cables 21, 22 by the locking components 13, 14. This other functionality of the pistons 211, 221 is realised, in the preferred embodiment of the invention, in the following way.

Each groove 1001 is made along its sliding guide 10 in an opening manner. Thus, each lug 2111, 2211 can extend, as shown in FIGS. 4D, by its distal end beyond the groove 1001. Moreover, each one of the two longitudinal portions 1311, 1312, 1411, 1412 of each locking component 13, 14 comprises, as shown in FIG. 6, a plurality of gripping devices 130, 140 each one configured to lock the distal end of a corresponding lug 2111, 2211, in such a way as to prevent the sliding thereof in its groove 1001 when the locking component 13, 14 is in the locking position. More particularly, in reference to FIG. 6 and to FIGS. 7 and 8, each gripping device 130, 140 comprises a removal of material having a first portion forming a secondary sliding groove 1301, 1401 of the distal end of the corresponding lug 2111, 2211 when the locking component 13, 14 is in the release position and a second portion forming a housing 1302, 1402 open only onto the secondary groove 1301, 14012 to house the distal end of the corresponding lug 2111, 2211 when the locking component 13, 14 is in the locking position.

As shown in FIG. 4D, each secondary groove 1301 of the first locking component 13 is configured to be aligned with a groove 1001 of a sliding guide when the locking component 13 is in the release position. In this way, the pistons 211, 221 connected in pairs remain free to slide in the sliding guides 10, when the locking component 13 is in the release position. The same applies to each secondary groove 1401 of the second locking component 14.

According to the preferred embodiment of the invention, each housing 1302, 1402 has, as shown in FIG. 6 and FIGS. 7 and 8, a flared shape section opening onto the secondary groove 1301, 1401 via its widest portion. And the latter has a width greater than the diameter of the lugs 2111, 2211. In this way, the locking of the distal portions of the lugs by the locking component thus configured benefits from a certain degree of freedom relative to the position of the distal end of the lug in the secondary groove. Consequently, the locking position of the first ends of the cables by the locking components benefit from the same degree of freedom.

Further note that each locking component 13, 14 comprises two longitudinal portions (1311, 1312, 1411, 1412), a first longitudinal portion 1311, 1411 comprising the gripping devices 130, 140 of the first ends of the cables 21, 22 intended for sliding in the plurality of sliding guides 10 made in the second component 115, 125 of the first and second blocks 11, 12 and a second longitudinal portion 1312, 1412 comprising the gripping devices 130, 140 of the first ends of the cables 21, 22 intended for sliding in the plurality of sliding guides 10 made in the third component 116, 126 of the first and second blocks 11, 12. The connector thus makes it possible to connect together twice as many pairs of cables together in a compact, simple and robust manner.

It stems, from the preceding description, the following operation of the connector for cables 1 according to the invention, and consequently of a cabled robot 9 provided with the connector for cables 1 according to the invention.

The connector for cables 1 is first of all set in place and the cabled robot 9 is calibrated. To do this, each cable 21 is inserted by its first end into one of the through-bores 100 of the first block 11 and each cable 22 is inserted by its first end into one of the through-bores 100 of the second block 12. The first end of each cable 21 is fastened to a piston 211 and the first end of each cable 22 is fastened to piston 221. The pistons 211, 221 are aligned in such a way as to be able to be locked by the locking components 13, 14. In the preferred embodiment of the invention, the lugs 2111 of the pistons 211 are then aligned together in the first block 11 and the lugs 2211 of the pistons 221 are then aligned together in the second block 12. The blocks 11, 12 are connected together. At this stage, the length of each one of the cables 21, 22, and in particular that of each one of the cables 22, is defined in such a way that the cabled robot 9, and more particularly an instrument or instrument element 93 of the cabled robot 9, is in a determined position that it will be able to return to in what follows before each disconnection of the connector for cables 1. Also at this stage, the tension of each pair of cables 21, 22, and in particular of each cable 21, can be calibrated in particular by determining an initial position of each one of the motorised rotors. The cabled robot 9 is ready for its first use. In order to correctly carry out the latter, the first and second locking components 13, 14 are displaced from their locking position to their release position. The control box 94 makes it possible to displace each pair of cables 21, 22 independently, as shown in FIG. 2B, of the others in order to control the instrument or instrument element 93 of the cabled robot 9. Once the first use has been correctly carried out, the instrument or instrument element 93 of the cabled robot 9 is brought back to its determined position corresponding to an alignment of the gripping devices 130, 140 of the locking components 13, 14 with the lugs 2111, 2211 of the pistons 211, 221, by return of the motorised rotors to their initial position. The locking components 13, 14 are brought back from their release position to their locking position. The blocks 11, 12 are then disengaged from one another, as shown in FIG. 2A. The portion of the cabled robot 9 comprising the second block 12, the cables 22 and the instrument or instrument element 93 can then be the object of a cleaning/sterilisation/storage independently of the rest of the cabled robot. Before a second use of the cabled robot 9, the instrument or instrument element 93 still being in its determined position, the two blocks 11, 12 are again connected together, before the locking components 13, 14 are displaced from their locking position to their release position. The calibration of the cabled robot 9 is thus retained identically with respect to the first use of the cabled robot 9.

Once the second use is correctly carried out, the locking components 13, 14 are again displaced from their release position to their locking position and the blocks 11, 12 are again disconnected from each other. Any subsequent use of the cabled robot 9 obeys the same kinematics making it possible to retain the initial calibration of the cabled robot.

The safety of the patients is thus assured, repeatably, by the retaining of the tension of use of the cables 21, 22 guaranteeing the retaining of the precision of the robot 9. The connection of blocks 11, 12 of the connector 1, making it possible simply and quickly to connect multiple cables simultaneously, is carried out unambiguously and in a minimised time, so as to respond to the temporal requirements of use of the robot 9, these requirements being for example linked to the anaesthesia of the patient and/or to the limited use time of an imaging device.

The invention is not limited to the embodiments described hereinabove and extends to all the embodiments covered by the claims.

### REFERENCES

(1) Connector

(10) Sliding guide

(100) Through-bore

(1001) Groove

(1002) Downstream portion

(1003) Upstream portion

(101) Connection face

(1011) First mouth

(102) Fastening face

(1021) First mouth

(11) First block

(111) Positioning shape

(112) Positioning pin

(113) Protuberance

(1131) Lateral side

(1132) Longitudinal side

(114) Fastening device

(115) Second block portion

(116) Third block portion

(12) Second block

(121) Positioning shape

(122) Drilling

(123) Protuberance

(1231) Lateral side

(1232) Longitudinal side

(124) Fastening device

(125) Second block portion

(126) Third block portion

(13) First locking component

(130) Gripping device

(1301) Secondary groove

(1302) Housing

(1311) Longitudinal portion

(1312) Longitudinal portion

(1313) First lateral portion

(1314) Second lateral portion

(14) Second locking component

(140) Gripping device

(1401) Secondary groove

(1402) Housing

(1411) Longitudinal portion

(1412) Longitudinal portion

(1413) First lateral portion

(1414) Second lateral portion

(151) First return member

(152) Second return member

(161) First latch

(162) Second latch

(21) First cable

(210) First end of the first cable

(211) Pistons

(2111) Lug

(2112) Annular groove

(22) Second cable

(220) First end of the second cable

(221) Piston

(2211) Lug

(2212) Collar

(8) Body of the patient

(9) Cabled robot

(91) Cable

(92) Motor

(93) Instrument

(94) Control box

1. A connector for cables comprising a first block and a second block, the first and second blocks being configured to be connected together and to form, when they are connected together, a plurality of sliding guides in each of which a pair of first and second cables, connected together at their respective first ends, is intended for sliding, the connector further comprising a first locking component mounted on the first block so as to be movable between a locking position and a release position of a set of first ends of the first cables, and the connector further comprising a second locking component mounted on the second block so as to be movable between a locking position and a release position of a set of first ends of the second cables.

2. The connector according to claim 1, wherein each block has at least two through-bores and a connection face to the other block, each through-bore of the first block opening by a first mouth from the connection face of the first block, each through-bore of the second block opening by a first mouth from the connection face of the second block, the first mouth of each one of said at least two through-bores of the first block being configured to be placed face to face with the first mouth of a respective one of said at least two through-bores of the second block, in such a way as to form said plurality of sliding guides when the first and second blocks are connected together.

3. The connector according to claim 1, wherein each one among the first and second locking components comprises at least one plurality of gripping devices each configured:
to engage the first end of one corresponding among the first and second cables, respectively, when the locking component is in its locking position, and
to release the first end of one corresponding among the first and second cables, respectively, when the locking component is in its release position.

4. The connector according to claim 3, wherein the gripping devices of each locking component are fixed with respect to their locking component and each locking component is configured to be moved in such a way as to induce a same movement of each one of its gripping devices, in such a way that the first ends of ones among the first and second cables are simultaneously engaged when the locking component passes from its release position to its locking position and in such a way that the first ends of ones among the first and second cables are simultaneously released when the locking component passes from its locking position to its release position.

5. The connector according to claim 1, further comprising a plurality of pistons each intended for being fastened to the first end of a cable, each piston fastened to the first end of the first cable of a pair of cables being configured to engage a piston fastened to the first end of the second cable of the pair, and wherein each sliding guide is configured in such a way that the first and second cables of the pair intended for sliding therein slide therein, at least by their pistons engaged together.

6. The connector according to claim 5, wherein each one among the first and second blocks are configured to be connected together by a relative connection movement of one with respect to the other, the pistons fastened to the first ends of the first cables and the pistons fastened to the first ends of the second cables being configured to be connected, in pairs, according to said relative movement, when the first and second locking components are in the locking position.

7. The connector according to claim 5, wherein at least one piston among the two pistons of a pair comprises on its outer periphery a lug extending substantially perpendicularly to a longitudinal direction of the sliding guide wherein it is configured to slide, and wherein each sliding guide comprises a groove extending continuously along the longitudinal direction of the sliding guide from the first block to the second block, when the first and second blocks are connected together, each groove being configured to guide the sliding of the lug.

8. The connector according to claim 7, wherein
each block has at least two through-bores and a connection face to the other block, each through-bore of the first block opening by a first mouth from the connection face of the first block, each through-bore of the second block opening by a first mouth from the connection face of the second block, the first mouth of each one of said at least two through-bores of the first block being configured to be placed face to face with the first mouth of a respective one of said at least two through-bores of the second block, in such a way as to form said plurality of sliding guides when the first and second blocks are connected together, and
each through-bore comprises, over a portion only of its extent, a portion of the groove of the sliding guide that it forms when the first and second blocks are connected together.

9. The connector according to claim 7, wherein each block has at least two through-bores and a connection face to the other block, each through-bore of the first block opening by a first mouth from the connection face of the first block, each through-bore of the second block opening by a first mouth from the connection face of the second block, the first mouth of each one of said at least two through-bores of the first block being configured to be placed face to face with the first mouth of a respective one of said at least two through-bores of the second block, in such a way as to form said plurality of sliding guides when the first and second blocks are connected together, and
wherein the pistons of a pair are configured to cooperate together by a fastening mechanism with annular groove and collar,
wherein, among the pistons each comprising a collar, the collars are configured according to a same specific orientation, for example opposite, with respect to the lugs of these pistons,
wherein the first and second locking components are configured to lock the first ends of the first and second cables respectively in such a way that each piston comprising an annular groove protrudes at least by its annular groove of the first and second blocks respectively, wherein each first mouth, configured to be placed face to face with a mouth from which the annular groove of a piston protrudes when the first and second locking components are in the locking position, has a bayonet fastening transverse section of the annular groove of the piston, and
wherein the grooves of the sliding guides are arranged according to a same orientation, in such a way that said relative movement of the first block with respect to the second block is a translation movement of one with respect to the other, which makes it possible to insert the annular groove of each piston of a pair into the collar of each piston of the pair.

10. The connector according to claim 1, wherein each one among the first and second blocks are configured to be connected together by a relative connection movement of one with respect to the other, and wherein the first block comprises at least one positioning shape configured to cooperate with a corresponding positioning shape of the second block, the positioning shapes of the first and second blocks cooperating together by a male-female adjustment mechanism configured to guide the relative connection movement of the first and second blocks together.

11. The connector according to claim 1, wherein each one among the first and second blocks is configured to be connected together by a relative connection movement of one with respect to the other and wherein one among the first and second blocks comprises a positioning pin configured to cooperate with a drilling comprised by the other among the first and second blocks, in such a way as to guide the relative connection movement of the first and second blocks together.

12. The connector according to claim 9, wherein
each one among the first and second blocks is configured to be connected together by a relative connection movement of one with respect to the other and wherein one among the first and second blocks comprises a positioning pin configured to cooperate with a drilling comprised by the other among the first and second blocks, in such a way as to guide the relative connection movement of the first and second blocks together, and
the positioning pin and the drilling cooperate together by a bayonet fastening mechanism that is equivalent, at least in terms of fastening movement amplitude and orientation, to the fastening mechanism with annular groove and collar according to which the pistons of a pair cooperate together.

13. The connector according to claim 1, wherein each one of the first and second blocks comprises a protuberance, the protuberance of the first block being in contact with the protuberance of the second block when the blocks are connected together, and wherein the first locking component is mounted on the protuberance of the first block so as to be movable in translation and the second locking component is mounted on the protuberance of the second block so as to be movable in translation.

14. The connector according to claim 13, wherein each protuberance comprises two lateral sides and two longitudinal sides and each locking component comprises at least one longitudinal portion and first and second lateral portions connected together by said at least one longitudinal portion, the protuberance of the first block being configured in such a way as to limit the translation movement of the first locking component to a continuum of positions comprised between its locking position and its release position by abutment of one among the first and second lateral portions of the first locking component on one of the two lateral sides of the protuberance of the first block and by abutment of the other among the first and second material portions of the first locking component on the other of the two lateral sides of the protuberance of the first block, and the protuberance of the second block being configured in such a way as to limit the translation movement of the second locking component to a continuum of positions comprised between its locking position and its release position by abutment of one among the first and second lateral portions of the second locking component on one of the two lateral sides of the protuberance of the second block and by abutment of the other among the first and second lateral portions of the second locking component on the other of the two lateral sides of the protuberance of the second block.

15. The connector according to claim 1, further comprising at least two return members, a first return member configured to exert on the first locking component a return force tending to maintain the first locking component in one of its locking and release positions, a second return member configured to exert on the second locking component a return force tending to maintain the second locking component in one of its locking and release positions, and the connector further comprising two latches,
a first latch being mounted on the first block so as to be movable between a retracted position and a deployed position, the first latch being configured jointly with the first locking component to oppose the return force that the first return member exerts on the first locking component in such a way as to maintain the first locking component in the other of its locking and release positions when the first latch is in its deployed position, and to not oppose the return force that the first return member exerts on the first locking component when the first latch is in its retracted position, and
a second latch being mounted on the second block so as to be movable between a retracted position and a deployed position, the second latch being configured jointly with the second locking component to oppose the return force that the second return member exerts on the second locking component in such a way as to maintain the second locking component in the other of its locking and release positions when the second latch is in its deployed position, and to not oppose the return force that the second return member exerts on the second locking component when the second latch is in its retracted position.

16. The connector according to claim 14, further comprising at least two return members, a first return member configured to exert on the first locking component a return force tending to maintain the first locking component in one of its locking and release positions, a second return member configured to exert on the second locking component a return force tending to maintain the second locking component in one of its locking and release positions, and the connector further comprising two latches,
a first latch being mounted on the first block so as to be movable between a retracted position and a deployed position, the first latch being configured jointly with the first locking component to oppose the return force that the first return member exerts on the first locking component in such a way as to maintain the first locking component in the other of its locking and release positions when the first latch is in its deployed position, and to not oppose the return force that the first return member exerts on the first locking component when the first latch is in its retracted position, and
a second latch being mounted on the second block so as to be movable between a retracted position and a deployed position, the second latch being configured jointly with the second locking component to oppose the return force that the second return member exerts on the second locking component in such a way as to maintain the second locking component in the other of its locking and release positions when the second latch is in its deployed position, and to not oppose the return force that the second return member exerts on the second locking component when the second latch is in its retracted position,
wherein the first latch is configured to be, in its deployed position, partially pinched between one of the lateral sides of the protuberance of the first block and the first lateral portion of the first locking component abutting on this lateral side when the first latch is in the retracted position and the first return member is arranged between the other of the lateral sides of the protuberance of the first block and the second lateral portion of the first locking component opposite the first lateral portion of the first locking component, and wherein the second latch is configured to be, in its deployed position, partially pinched between one of the lateral sides of the protuberance of the second block and the first lateral portion of the second locking component abutting on this lateral side when the second latch is in the retracted position and the second return member is arranged between the other of the lateral sides of the protuberance of the second block and the second lateral portion of the second locking component opposite the first lateral portion of the first locking component.

17. A connector for cables comprising a pair of first and second cables and a first and a second locking components,
wherein each one among the first and second locking components comprises at least one plurality of gripping devices each configured:
to engage a first end of one corresponding among the first and second cables, respectively, when the locking component is in its locking position, and
to release the first end of one corresponding among the first and second cables, respectively, when the locking component is in its release position;
wherein at least one piston among the two pistons of a pair comprises on its outer periphery a lug extending substantially perpendicularly to a longitudinal direction of a plurality of sliding guides wherein the sliding guides are configured to slide, and wherein each sliding guide comprises a groove extending continuously along the longitudinal direction of the sliding guide from a first block to a second block, when the first and second blocks are connected together, each groove being configured to guide the sliding of the lug;
wherein each protuberance of each one of the first and second blocks further comprises two lateral sides and two longitudinal sides and each locking component comprises at least one longitudinal portion and first and second lateral portions connected together by said at least one longitudinal portion, the protuberance of the first block being configured in such a way as to limit the translation movement of the first locking component to a continuum of positions comprised between its locking position and its release position by abutment of one among the first and second lateral portions of the first locking component on one of the two lateral sides of the protuberance of the first block and by abutment of the other among a first and second material portions of the first locking component on the other of the two lateral sides of the protuberance of the first block, and the protuberance of the second block being configured in such a way as to limit a translation movement of the second locking component to a continuum of positions comprised between its locking position and its release position by abutment of one among the first and second lateral portions of the second locking component on one of the two lateral sides of the protuberance of the second block and by abutment of the other among the first and second lateral portions of the second locking component on the other of the two lateral sides of the protuberance of the second block; and
wherein each groove opens in such a way that each lug extends by its distal end beyond the groove configured to guide the sliding thereof, and wherein said at least one longitudinal portion of each locking component comprises one among said at least one plurality of gripping devices, each gripping device being configured to lock the distal end of a corresponding lug in such a way as to prevent the sliding thereof in its groove when the locking component is in the locking position.

18. The connector according to claim 17, wherein each gripping device comprises a removal of material made in said at least one longitudinal portion of the locking component and having a first portion forming a secondary sliding groove of the distal end of the corresponding lug when the locking component is in the release position and a second portion forming a housing open only onto the secondary groove to house the distal end of the corresponding lug when the locking component is in the locking position.

19. The connector according to claim 18, wherein each housing has, in a longitudinal section plane of the longitudinal portion of the locking component, a flared shape section opening onto the secondary groove via its widest portion, the latter having a width greater than a diameter of the distal end of the lug that it houses when the locking component is in its locking position.

20. The connector according to claim 1, wherein each block comprises a fastening device to the other block, the fastening device of the first block being configured to removably engage the fastening device of the second block, in such a way as to maintain the first and second blocks connected together, at least when the first ends of the cables are connected in pairs and the locking components are in their release position.

21. The connector according to claim 14, wherein each block comprises a second block portion and a third block portion removably fastened together, each second block portion comprising a downstream portion of a first plurality of sliding guides and each third block portion comprising an upstream portion of a second plurality of sliding guides, wherein one of the second and third block portions comprises the protuberance of the block and wherein each locking component comprises two longitudinal portions, each one connected to opposite ends of the lateral portions of the locking component, a first longitudinal portion comprising the gripping devices of the first ends of the cables intended for sliding in the first plurality of sliding guides and a second longitudinal portion comprising the gripping devices of the first ends of the cables intended for sliding in the second plurality of sliding guides.

22. A cabled robot comprising the connector for cables according to claim 1.
